(19) United States
(12) Patent Application Publication
(10) Pub. No.: US 2013/0231752 A1
(43) Pub. Date: Sep. 5, 2013

(54) ENDOLUMINAL PROSTHESIS WITH ACTUATING MEMBER

(71) Applicants: Joanna Lynn Rosenbaum, Bloomington, IN (US); Benjamin T. Biltz, Spencer, IN (US)

(72) Inventors: Joanna Lynn Rosenbaum, Bloomington, IN (US); Benjamin T. Biltz, Spencer, IN (US)

(73) Assignee: Cook Medical Technologies LLC, Bloomington, IN (US)

(21) Appl. No.: 13/410,663

(22) Filed: Mar. 2, 2012

(51) Int. Cl. A61F 2/04

(52) U.S. Cl. 623/23.7

(57) ABSTRACT

A non-expandable endoluminal prosthesis for implantation within a body lumen may include an elongate tubular conduit. The tubular conduit may have a first end segment and a second end segment. A drainage lumen may extend longitudinally within the tubular conduit. An actuating lumen may extend longitudinally within the tubular conduit. The prosthesis may include an actuating member received within the actuating lumen. The endoluminal prosthesis may be movable between a delivery configuration in which the tubular conduit is substantially linear and a deployed configuration in which at least one of the first end segment and the second end segment includes a retaining mechanism configured to retain the prosthesis in place relative to the body lumen. The actuating member may be configured to urge the prosthesis toward the deployed configuration.

### TECHNICAL FIELD

This disclosure relates generally to medical devices. More specifically, this disclosure relates to drainage devices such as ureteral stents useful for urinary drainage.

### BACKGROUND

Indwelling ureteral stents are in common use today. These stents are placed in the ureter, which is the duct between the kidney and the bladder, for establishing and/or maintaining an open, patent passageway for the flow of urine from the kidney to the bladder. The predominate indications for placing a ureteral stent include extrinsic compression, ureteral injury due to trauma, obstructive uropathy, and following surgery in the upper or lower urinary tract. Generally, the stent includes a flexible material having sufficient resiliency to allow the stent to be straightened for insertion into the ureter, while having sufficient memory to return to a predetermined retentive shape when in situ.

Indwelling ureteral stents may be positioned in the ureter by one of a variety of procedures including, antegrade (percutaneous) placement, retrograde (cystoscopic) placement through the urethra, placement by open ureterotomy, or surgical placement in the ureter under direct visual placement. Ureteral stent positioning may be accomplished by several methods. In one method, a wire guide is introduced into the ureteral orifice in the bladder via a cystourethroscope under direct vision. The wire guide is advanced up the ureter until the advancing flexible tip of the guide is confirmed by x-ray or fluoroscopy to be in the renal pelvis of the kidney. A tubular stent with both ends open is fed into the exposed external segment of the wire guide and advanced over the wire guide by hand until a short segment of the stent is visible outside the cystourethroscope. A positioner, pusher catheter, or length of tubing is then fed into the exposed external end of the wire guide and advanced over the wire guide by hand until it abuts the stent. With the wire guide held stationary, the positioner is advanced over the wire guide to push the tubular stent up the ureter to the renal pelvis. With the distal end of the stent in the renal pelvis, the positioner is held stationary while the wire guide is gradually extracted from the stent and the positioner. As the wire guide leaves the distal end of the tubular stent, a retentive means at the distal end of the stent is formed to retain the stent in the pelvis of the kidney. As the wire guide is withdrawn past the proximal end of the stent, a retentive hook or curve at the proximal end is formed so that the stent is retained within the bladder. At this point, the positioner and wire guide are completely withdrawn leaving only the stent indwelling in the kidney, the ureter, and the bladder.

In another method of ureteral stent placement, a ureteral stent having one tip closed is backloaded into the wire guide. In this “pushup” method, the tip of the wire guide contacts the closed tip of the ureteral stent, which is then introduced into the ureteral orifice in the bladder via a cystourethroscope under direct vision. The stent is advanced up the ureter until the tip of the stent lies within the renal pelvis. A positioner catheter or length of tubing is fed into the external end of the wire guide and advanced over the wire guide by hand until it abuts the open end of the stent. The positioner is held in place while removing the wire guide to leave the stent positioned within the ureter.

In some cases, the ureteral stent may be associated with pain or discomfort for the patient. For example, such pain or discomfort may be caused by a failure of the stent to conform to the patient's ureteral anatomy. This pain or discomfort may be exacerbated by physical movement, respiration, or bladder contractions and expansions of the patient.

### SUMMARY

The present embodiments provide a drainage device such as a ureteral stent useful for urinary drainage.

In one example, a non-expandable endoluminal prosthesis for implantation within a body lumen may include an elongate tubular conduit. The tubular conduit may have a first end segment and a second end segment. A drainage lumen may extend longitudinally within the tubular conduit. An actuating lumen may extend longitudinally within the tubular conduit. The prosthesis may include an actuating member received within the actuating lumen. The endoluminal prosthesis may be movable between a delivery configuration in which the tubular conduit is substantially linear and a deployed configuration in which at least one of the first end segment and the second end segment includes a retaining mechanism configured to retain the prosthesis in place relative to the body lumen. The actuating member may be configured to urge the prosthesis toward the deployed configuration.

In another example, a system may include a non-expandable endoluminal prosthesis for implantation within a body lumen and a guide wire. The prosthesis may include an elongate tubular conduit. The tubular conduit may have an end segment. A drainage lumen may extend longitudinally within the tubular conduit. An actuating lumen may extend longitudinally within the tubular conduit. The prosthesis may include an actuating member received within the actuating lumen. The actuating member may be movable between a relaxed condition in which a longitudinal axis of the actuating member is substantially non-linear and a strained condition in which the longitudinal axis of the actuating member is substantially linear. The guide wire may be receivable within the drainage lumen of the prosthesis. With the guide wire received within a portion of the drainage lumen corresponding to the end segment of the tubular conduit, the actuating member may be in the strained condition. Upon removal of the guide wire from the portion of the drainage lumen corresponding to the end segment, the actuating member may move to the relaxed condition to form a retaining mechanism in the end segment of the tubular conduit.

In another example, a method of implanting an endoluminal prosthesis within a body lumen may include introducing the endoluminal prosthesis in a delivery configuration into the body lumen over a wire guide. The endoluminal prosthesis may include an elongate tubular conduit having a first end segment and a second end segment. A drainage lumen may extend longitudinally within the tubular conduit. An actuating lumen may extend longitudinally within the tubular conduit. An actuating member may be received within the actuating lumen. The method may include removing the wire guide from the first end segment of the tubular conduit to enable at least a first portion of the actuating member corresponding to the first end segment to move to a relaxed configuration to form a first retaining mechanism in the first end segment of the tubular conduit. The method may include removing the wire guide from the second end segment of the tubular conduit to enable at least a second portion of the actuating member corresponding to the second end segment to move to the relaxed configuration to form a second retaining mechanism in the second end segment of the tubular conduit.

Other systems, methods, features, and advantages of the invention will be, or will become, apparent to one with skill in the art upon examination of the following figures and detailed description. It is intended that all such additional systems; methods, features, and advantages be within the scope of the invention, and be encompassed by the following claims.

### BRIEF DESCRIPTIONS OF THE DRAWINGS

FIG. 1 illustrates one embodiment of a drainage device configured as a ureteral stent.

FIG. 1A is a close-up view of a distal end segment of the drainage device of FIG. 1.

FIG. 1C is a transverse cross sectional view of a distal end segment of the drainage device of FIG. 1.

FIG. 2 illustrates another embodiment of a drainage device configured as a ureteral stent.

FIG. 2B is a close-up view of a distal end segment of the drainage device of FIG. 2.

FIG. 3 illustrates a drainage device in a delivery configuration on a wire guide.

FIG. 4A illustrates a illustrates the introduction of a drainage device into a ureter of a patient.

FIG. 4B illustrates a drainage device indwelling within a ureter of a patient.

### DETAILED DESCRIPTION OF THE DRAWINGS AND THE PRESENTLY PREFERRED EMBODIMENTS

Detailed embodiments of the present invention are disclosed herein. It is understood, however, that the disclosed embodiments are merely exemplary of the invention, which may be embodied in various and alternative forms. The figures are not necessarily to scale, and some figures may be configured to show the details of a particular component. Therefore, specific structural and functional details disclosed herein are not to be interpreted as limiting, but merely as a representative basis for the claims and for teaching one skilled in the art to practice the present invention.

In the present disclosure, the term “proximal” refers to a direction that is generally toward a physician during a medical procedure, while the term “distal” refers to a direction that is generally toward a target site within a patient's anatomy during a medical procedure.

Various medical devices for implantation in a body vessel are disclosed herein. Preferred embodiments relate to a medical drainage device including one or more actuating members configured to move at least a portion of the drainage device from a delivery configuration to a deployed configuration. The medical drainage devices are described with respect to an exemplary ureteral stent embodiment including a tubular conduit. However, this disclosure is not so limited, and may be applicable to other medical drainage devices such as biliary stents, esophageal stents, or other types of drainage devices. For example, a drainage stent may be configured for use within a biliary, pancreatic, urethral, esophageal, or blood vessel. In any of the embodiments described herein, the medical drainage device may be configured as an expandable or a non-expandable endoluminal prosthesis.

FIG. 1 shows one embodiment of a medical drainage device 100 configured as a ureteral stent. The drainage device 100 may include a tubular conduit 110. The tubular conduit 110 may be configured as an elongate tubular member having an inlet 112 and an outlet 114. A drainage lumen 116 may extend longitudinally within the tubular conduit 110 between the inlet 112 and the outlet 114. The drainage lumen 116 may be in fluid communication with the inlet 112 and the outlet 114 to provide a continuous passageway for fluid to flow through the drainage device 100. In any of the embodiments described herein, the tubular conduit 110 may include one or more drainage holes 117. Each drainage hole 117 may extend through a wall of the tubular conduit 110, and the drainage lumen 116 may be in fluid communication with a point external of the tubular conduit through the drainage hole. Fluid may be allowed to flow through the drainage holes 117 to enhance the flow of fluid through the drainage device 100. In other embodiments, the inlet 112 and/or the outlet 114 may be omitted, and fluid may enter or exit the drainage lumen 116 through the drainage holes 117.

The tubular conduit 110 may be formed from any suitable material known in the art. For example, the tubular conduit 110 may be formed from silicone, polyurethane, polyamide, polyvinyl chloride, or any other suitable polymer or metal. The tubular conduit 110 may be a solid structure or a porous structure. In one preferred embodiment, the tubular conduit 110 may be formed from expanded polytetrafluoroethylene (ePTFE) tubing. Such an ePTFE material may be highly flexible and lubricious. When fabricated in an appropriate porosity range, the ePTFE tubing also may exhibit sufficient column strength to withstand introduction within a body vessel and sufficient radial compression strength to maintain an open lumen (e.g., the drainage lumen 116) upon implantation within the body vessel. In one example, the porosity of the ePTFE tubing may range from about 5 to about 60 microns. Additionally, the ability of the ePTFE tubing to conform to the patient's anatomy may make the drainage device 100 having the tubular conduit 110 formed from ePTFE particularly comfortable for the patient when implanted within the body lumen.

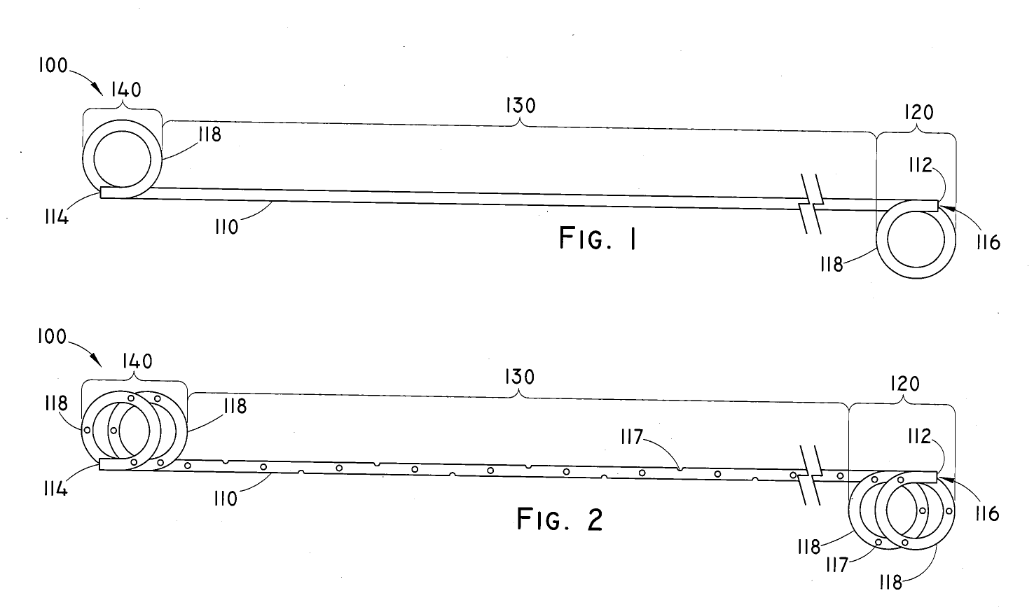

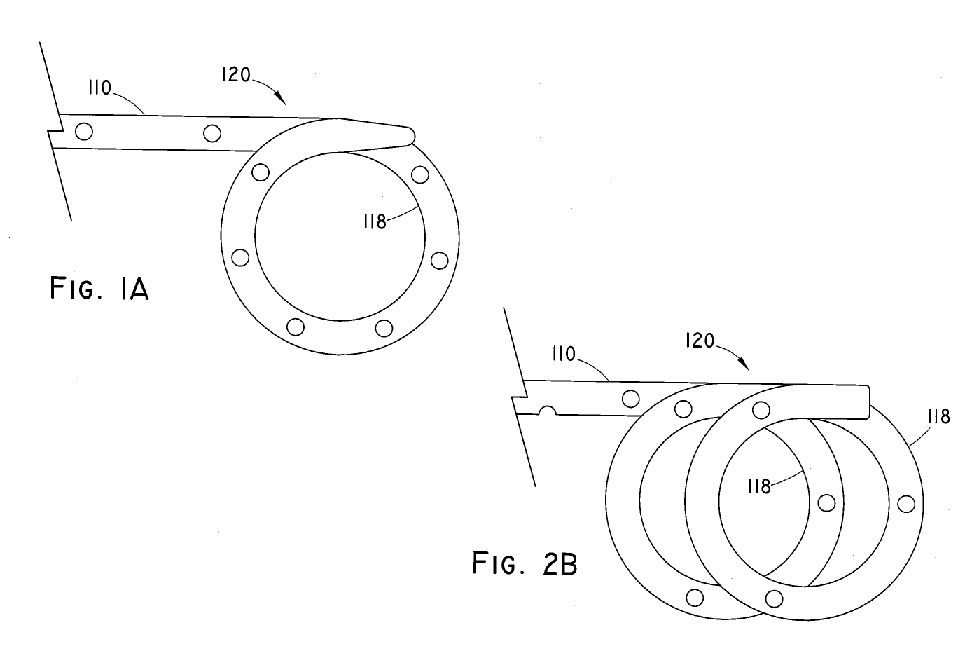

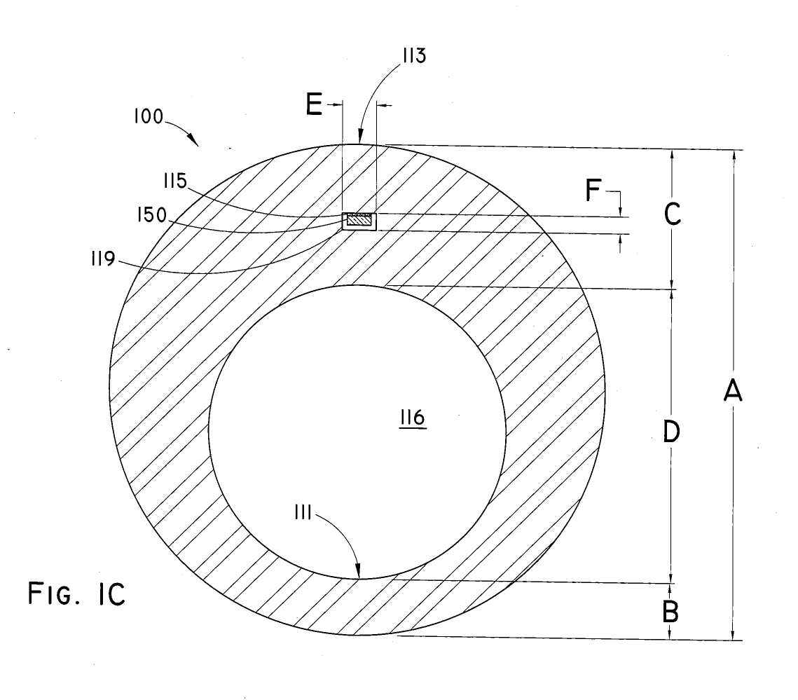

The drainage device 100 may include a distal end segment 120, a proximal end segment 140, and an intermediate segment 130 adjoining the distal end segment and the proximal end segment to one another. The distal end segment 120 and/or the proximal end segment 140 may be configured as a retaining mechanism for retaining the drainage device 100 in a desired location within a body lumen (e.g., the ureter). To that end, the distal end segment 120 and/or the proximal end segment 140 of the drainage device 100 may include one or more loops 118 formed in the tubular conduit 110. For example, each of the distal end segment 120 and the proximal end segment 140 may include one loop 118 formed in the tubular conduit 110 as shown in FIGS. 1-1A. This may be referred to as a double-J pigtail configuration. In another example, each of the distal end segment 120 and the proximal end segment 140 may include two loops 118 formed in the tubular conduit 110 as shown in FIGS. 2-2B. This may be referred to as a multi-length configuration. In other examples, the distal end segment 120 and the proximal end segment 140 may include any number of loops 118. The distal end segment 120 may include the same or a different number of loops 118 as the proximal end segment 140. The loops 118 may be configured to retain the corresponding end segment of the drainage device 100 in a desired location within the body (e.g., a kidney or a bladder) as further described below. In other examples, the retaining mechanism of the distal end segment 120 and/or the proximal end segment 140 may be J shaped, helical shaped, zig-zag shaped, or any other shape configured to retain the drainage device 100 in a desired location within a body lumen. Additionally, or alternatively, the distal end segment 120 and/or the proximal end segment 140 may include a barb, a malecot, a balloon, or any other suitable mechanism configured to retain the drainage device 100 in a desired location within a body lumen. In any of the examples described herein, a retaining mechanism having any known configuration may be substituted for the loops 118 without departing from the scope of this disclosure.

FIG. 1C shows a transverse cross sectional view of the distal end segment 120 of the drainage device 100. The tubular conduit 110 of the drainage device 100 may include an actuating lumen 119. The actuating lumen 119 may extend longitudinally within the tubular conduit 110. In one example, the actuating lumen 119 may extend along substantially an entire length of the tubular conduit 110 between the inlet 112 and the outlet 114. In another example, the actuating lumen 119 may extend along a portion of the length of the tubular conduit 110 corresponding to the distal end segment 120 or the proximal end segment 140. In yet another example, the actuating lumen 119 may be configured as two actuating lumens: a distal actuating lumen extending along a portion of the length of the tubular conduit 110 corresponding to the distal end segment 120 and a proximal actuating lumen extending along a portion of the length of the tubular conduit corresponding to the proximal end segment 140. The actuating lumen 119 may be configured to receive an actuating member as further described below to form the loops 118 in the tubular conduit 110.

The tubular conduit 110 may be configured as a length of dual lumen tubing. The dual lumen tubing may be formed using any process known in the art including, for example, extrusion. The actuating lumen 119 may be positioned adjacent to the drainage lumen 116 within the tubular conduit 110. To that end, the drainage lumen 116 may be offset from the longitudinal axis of the tubular conduit 110. In other words, the tubular conduit 110 and the drainage lumen 116 may not be coaxial with one another. For example, the drainage lumen 116 may be offset within the tubular conduit 110 so that the tubular conduit includes a thin walled section 111 and a thick walled section 113. The thickness of the wall of the tubular conduit 110 may taper circumferentially around the tubular conduit from a minimum thickness at the thin walled section 111 to a maximum thickness at the thick walled section 113 as shown in FIG. 1C. The actuating lumen 119 may be positioned within the thick walled section 114 of the tubular conduit 110. In other examples, the drainage lumen 116 may be coaxial with the tubular conduit 110, and the actuating lumen 119 may be positioned adjacent to the drainage lumen 116 within the tubular conduit.

The actuating lumen 119 may have any suitable cross sectional shape. The cross sectional shape of the actuating lumen 119 may be configured to correspond to a cross sectional shape of an actuating member 150 which may be received in the actuating lumen as further described below. For example, the actuating lumen 119 may have a rectangular cross sectional shape as shown in FIG. 1C. Such a rectangular actuating lumen 119 may be configured to receive an actuating member also having a rectangular cross sectional shape. In other examples, the actuating lumen 119 may have a circular, elliptical, triangular, arcuate, or any other polygonal or non-polygonal shaped cross section.

The drainage device 100 also may include at least one actuating member 150. The actuating member 150 may be received within the actuating lumen 119 as shown in FIG. 1C. The actuating member 150 may extend along substantially an entire length of the tubular conduit 110 between the inlet 112 and the outlet 114 of the drainage device 100. In other examples, the drainage device may include one actuating member received in a portion of the actuating lumen 119 corresponding to the distal end segment 120 of the drainage device 100 and another actuating member received in a portion of the actuating lumen 119 corresponding to the proximal end segment 140 of the drainage device. In still other examples, the drainage device may include multiple actuating members received in multiple actuating lumens as described above.

The actuating member 150 may be configured to form the loops 118, or a retaining mechanism having any other shape, in the tubular conduit 110 as described above. To that end, the actuating member 150 may include a shape memory material or a superelastic material. The actuating member 150 may include any shape memory or superelastic material including, for example, a shape memory or superelastic metal such as nitinol (i.e. a nickel-titanium alloy), stainless steel, copper-zinc-aluminum-nickel alloy, copper-aluminum-nickel alloy, or any other alloy which may include zinc, copper, gold, and/or iron. The actuating member 150 may include any shape memory polymer such as, for example, polyurethane, polyether ether ketone (PEEK), polyethylene terephthalate (PET), polyethylene oxide (PEO), polystyrene, or copolymers thereof. In one example, the actuating member 150 may be configured as a length of wire formed of a superelastic metal. In this example, the wire may be formed from a superelastic nitinol alloy, which may exhibit stress induced martensite at a body temperature. In other examples, the wire may be formed from a shape memory nitinol alloy, which may exhibit temperature induced martensite at a body temperature. The wire may have a rectangular cross sectional shape as shown in FIG. 1C. In other words, the wire may have a conventional flat wire configuration. Such a flat wire configuration may reduce the required profile, or height, of the actuating lumen 119. In other examples, the wire may have an arcuate cross-sectional shape. The arcuate cross-sectional shape may have a curvature similar to a curvature of the outer wall of the tubular conduit 110. In other examples, the wire may have a circular elliptical, triangular, or any other polygonal or non-polygonal shaped cross section.

The nitinol alloys described herein may exhibit superelastic or shape memory behavior. That is, the nitinol alloy may undergo a reversible phase transformation that allows it to “remember” and return to a previous shape or configuration. The nitinol alloy may transform between a lower temperature phase (martensite) and a higher temperature phase (austenite). Austenite is characteristically the stronger phase, and martensite may be deformed up to a recoverable strain of about 8%. Strain introduced in the alloy in the martensitic phase to achieve a shape change may be substantially recovered upon completion of a reverse phase transformation to austenite, allowing the alloy to return to a previous shape. The strain recovery may be driven by the application and removal of stress (superelastic effect) and/or by a change in temperature (shape memory effect).

The actuating member 150 also may enhance the structural stability of the drainage device 100. For example, the actuating member 150 may be substantially inflexible in the direction of the longitudinal axis of the tubular conduit 110. Upon application of a force to the drainage device 100 in the direction of the longitudinal axis of the tubular conduit 110, the actuating member 150 may not bend or flex to a substantial degree. This longitudinal rigidity may help to prevent buckling or folding (e.g., like an accordion) of the tubular conduit 110 of the drainage device 100 upon implantation in the patient's body.

The actuating member 150 may be retained within the actuating lumen 119 by a friction or interference fit. In one example, one or both ends of the actuating lumen 119 may be closed (e.g., using a tipping operation) to retain the actuating member 150 within the actuating lumen. In other words, the actuating lumen 119 may be configured as a chamber within the drainage device 100 and sealed on either end. In this example, the drainage device 100 may include a cap which may be bonded to the end of the drainage device to close the lumen 119. Alternatively, or additionally, the drainage device 100 may include an adhesive 115 disposed between an inner surface of the actuating lumen 119 and the actuating member 150. The adhesive 115 may be disposed between one surface of the actuating member 150 and the inner surface of the actuating lumen 119 as shown in FIG. 1C. In other examples, the adhesive 115 may substantially surround the actuating member 150 to fill a gap which may be formed between the actuating member and the inner surface of the actuating lumen 119. The adhesive 115 may include any biocompatible adhesive material known in the art. For example, the adhesive 115 may include a cyanoacrylate (e.g., ethyl cyanoacrylate, butyl cyanoacrylate, octyl cyanoacrylate, and hexyl cyanoacrylate), an epoxy, a silicone, or any combination thereof. Additionally, or alternatively, the tubular conduit 110 and/or the actuating member 150 may be crimped using any suitable crimping technique to retain the actuating member within the actuating lumen 119.

The actuating member 150 may be configured such that, in a relaxed condition, the actuating member takes on the desired shape of the retaining mechanism (e.g., the loops 118). The actuating member 150 may be formed into the desired shape by any means known in the art. For example, the loops 118 may be formed in the actuating member 150 using a shape setting process such as heating in a media bath (e.g., a salt bath or a sand bath), heating in an oven (e.g., an air furnace or a vacuum furnace), heating on a heated die, cold working, stamping, injection molding, exposure to infrared (IR) radiation, or exposure to radio frequency (RF) energy. The actuating member 150, received within the actuating lumen 119, may cause the distal end segment 120 and/or the proximal end segment 140 of the tubular conduit 110 to take on the shape of the actuating member. In this manner, the actuating member 150 may form the loops 118 in the tubular conduit 110.

The tubular conduit 110 of the drainage device 100 may be formed from an ePTFE material as described above. This ePTFE may exhibit desirable column strength and radial compression strength. However, the ePTFE may not exhibit shape memory or superelastic properties. In other words, ePTFE may be substantially unable to return to a predefined shape following deformation. The presence of the actuating member 150 within the actuating lumen 119 of the drainage device 100 may enable the tubular conduit 110 formed from ePTFE to exhibit the desired shape memory or superelastic properties. In other words, including the actuating member 150 within the actuating lumen 119 may provide the tubular conduit 110 with the desired shape memory or superelastic properties (due to the superelasticity or shape memory properties of the actuating member 150) so that the loops 118 may be formed in the distal end segment 120 and/or the proximal end segment 140 of the tubular conduit even though the ePTFE material itself may not exhibit such shape memory or superelastic properties. Because the ePTFE itself may not be required to exhibit shape memory or superelastic properties, the durometer, or hardness, of the tubular conduit 110 may be reduced relative to conventional drainage devices. In one example, the durometer of the tubular conduit 110 may range from about 15 to about 90 measured on a type A scale. In other words, the presence of the actuating member 150 may enable a softer tubular conduit 110 to be used. This softer tubular conduit 110 may be more comfortable for the patient upon implantation of the drainage device 100 including ePTFE.

The tubular conduit 110 may be configured as a length of dual lumen tubing as described above. The tubular conduit 110 may be sized and shaped for implantation within a body lumen. Exemplary dimensions of the tubular conduit 110 are described below with reference to FIG. 1C. The dimensions are merely exemplary and not limiting. The tubular conduit 110 may have any dimensions suitable for the intended use of the drainage device 100. In one example, the tubular conduit 110 may be formed from a length of 4.7 Fr dual lumen tubing. In this example, the tubular conduit 110 may have an outer diameter A of about 0.062 inches. The thin walled section 111 of the tubular conduit 110 may have a thickness B of about 0.005 inches, and the thick walled section 113 may have a thickness C of about 0.014 inches. The drainage lumen 116 may have a diameter D of about 0.043 inches. The actuating lumen 119 may have a rectangular cross section, as described above, having a width E of about 0.012 inches and a height F of about 0.004 inches. In another example, the tubular conduit 110 may be formed from a length of 6 Fr dual lumen tubing. In this example, the outer diameter A of the tubular conduit 110 may be about 0.079 inches. The thickness B of the thin walled section 111 may be about 0.009 inches, and the thickness of the thick walled section 113 may be about 0.023 inches. The diameter D of the drainage lumen 116 may be about 0.047 inches. In yet another example, the tubular conduit 110 may be formed from a length of 7 Fr dual lumen tubing. In this example, the outer diameter A of the tubular conduit 110 may be about 0.092 inches. The thickness B of the thin walled section 111 may be about 0.010 inches, and the thickness of the thick walled section 113 may be about 0.026 inches. The diameter D of the drainage lumen 116 may be about 0.056 inches. In still another example, the tubular conduit 110 may be formed from a length of 8 Fr dual lumen tubing. In this example, the outer diameter A of the tubular conduit 110 may be about 0.105 inches. The thickness B of the thin walled section 111 may be about 0.012 inches, and the thickness of the thick walled section 113 may be about 0.038 inches. The diameter D of the drainage lumen 116 may be about 0.055 inches. In other examples, the tubular conduit 110 may be formed from a length of dual lumen tubing having any size such as, for example, about 2 to about 26 Fr. The dual lumen tubing may be sized for placement at a particular location within a patient's body.

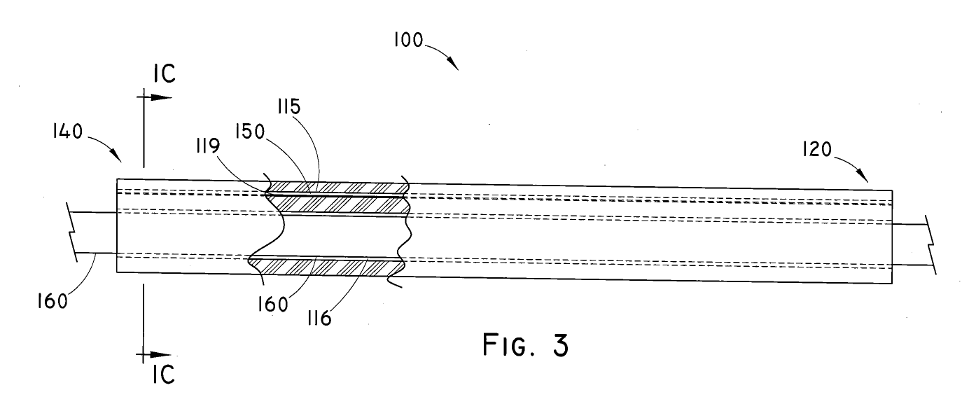

The drainage device 100 may be movable between a delivery configuration and a deployed configuration. FIG. 3 shows the drainage device 100 in the delivery configuration. In the delivery configuration, the tubular conduit 110 of the drainage device 100 may be substantially linear. In other words, the loops 118 of the distal end segment 120 and the proximal end segment 140 may be unrolled or straightened so that the tubular conduit is substantially linear as shown in FIG. 3. A wire guide 160 may be received within the drainage lumen 116 of the drainage device 100. The wire guide 160 may restrain the tubular conduit 110 of the drainage device in the delivery configuration. In other words, the wire guide 160 received within the drainage lumen 116 may counteract the force of the actuating member 150 received within the actuating lumen 119 to prevent the loops 118 from being formed in the distal end segment 120 and the proximal end segment 140. Upon removal of the wire guide 160 from the drainage lumen 116, the actuating member 150 may cause the drainage device 100 to return to the deployed configuration as shown in FIG. 1.

Implantation of the drainage device will be described in further detail below. Although the description will generally refer to the implantation of a ureteral stent within a ureter, a similar method may be used to implant a drainage device in any other body lumen.

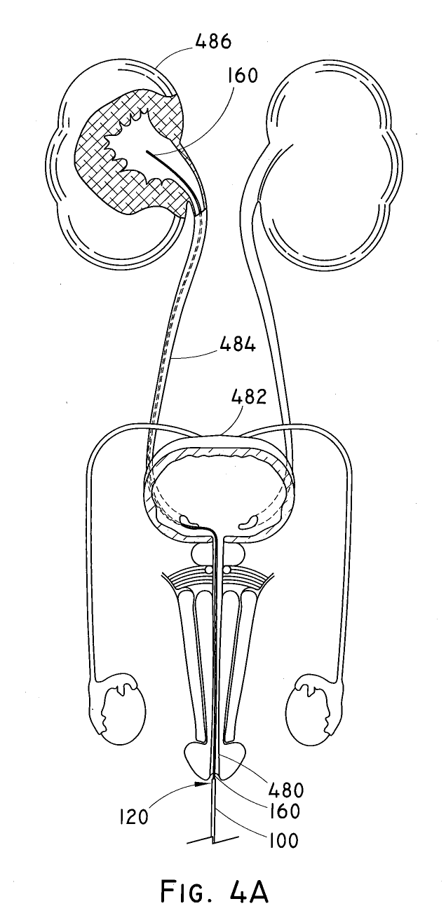

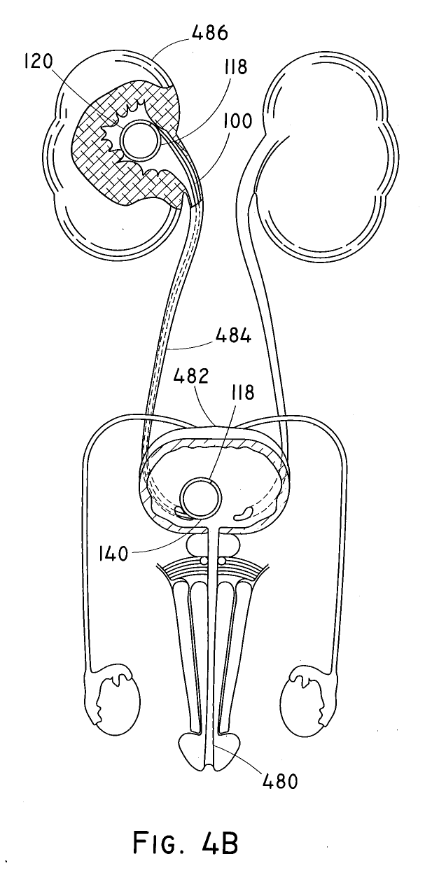

The wire guide 160 may be introduced into a urethra 480 of a patient as shown in FIG. 4A. The wire guide 160 may be advanced through the urethra 480 and into the bladder 482. The wire guide 160 then may be advanced into a ureter 484 and to a kidney 486. A cytoscope or other visualization device may be used to aid in positioning the wire guide 160. The position of the distal end of the wire guide 160 in the kidney 486 may be confirmed by x-ray, fluoroscopy, ultrasound, or any other suitable visualization technique.

The drainage device 100 may be advanced over the proximal end of the wire guide 160 remaining outside of the patient's body. The wire guide 160 may be received within the drainage lumen 116 of the drainage device 100 to restrain the drainage device in the delivery configuration as described above. The drainage device 100 may be advanced over the wire guide 160 and through the urethra 480, the bladder 482, and the ureter 484. The drainage device 100 may be advanced until the distal end segment 120 of the drainage device is positioned within the kidney 486. The drainage device 100 may be advanced by hand (i.e., by pushing the drainage device along the wire guide 160) until the proximal end segment 140 of the drainage device is near the end of the urethra. Then, the drainage device 100 may be advanced using a positioner. For example, the positioner may be advanced over the wire guide 160 until the distal end of the positioner contacts the proximal end segment 140 of the drainage device 100. The positioner may be further advanced to push the drainage device 100 along the wire guide 160. When the drainage device 100 is in the desired position within the ureter 484, the positioner may be retracted from the patient's body.

With the distal end segment 120 of the drainage device 100 positioned within the kidney 486, the wire guide 160 may be retracted proximally relative to the drainage device. Upon removal of the wire guide 160 from the portion of the drainage lumen 116 corresponding to the distal end segment 120 of the drainage device 100, the actuating member 150 may cause the loop 118, or other retention mechanism, to be formed in the distal end segment as described above. The loop 118 may have a diameter that is larger than a diameter of the ureter 484. In this manner, the loop 118 may retain the distal end segment 120 of the drainage device 100 within the kidney 486. In other words, the loop 118 may prevent the drainage device from moving within the ureter 484 away from the kidney 486 and toward the bladder 482. Additionally, or alternatively, a change in the temperature of the actuating member 150 may cause the loop 118 to be formed in the distal end segment 120 of the drainage device 100. In other words, the loops 118 may be formed in response to exposure to a body temperature within the patient's body (e.g., for shape memory alloys or polymers). The wire guide 160 may be further retracted to remove the wire guide from the drainage device. Upon removal of the wire guide 160 from the portion of the drainage lumen 116 corresponding to the proximal end segment 140 of the drainage device 100, the actuating member 150 may cause the loop 118, or other retention mechanism, to be formed in the proximal end segment as described above. The loop 118 may have a diameter that is larger than a diameter of the ureter 484. In this manner, the loop 118 may retain the proximal end segment 140 of the drainage device 100 within the bladder 482. In other words, the loop 118 may prevent the drainage device from moving within the ureter 484 away from the bladder 482 and toward the kidney 486. FIG. 4B shows the drainage device 100 in the deployed configuration (i.e., with the retaining mechanisms formed at the distal end segment 120 and the proximal end segment 140 of the drainage device) implanted within the ureter 484. The drainage device 100 may enable a fluid such as urine to flow through the drainage lumen 116 from the kidney 486 to the bladder 482. The intermediate segment 140 of the drainage device 100 may aid in maintaining the patency of the ureter 484.

Forming the tubular conduit 110 of the drainage device 100 from ePTFE, as described above, may enhance the conformance of the drainage device to the patient's ureteral anatomy. This enhanced conformance may be enabled by the highly flexible nature of the ePTFE. The ePTFE also may exhibit superior lubricity relative to other polymers. For example, the drainage device 100 including ePTFE may have a lower coefficient of friction against the wire guide 116 compared to conventional drainage devices including polyurethane. In one example, the coefficient of friction for ePTFE may range from about 0.02 to about 0.2. The coefficient of friction for polyurethane may range from about 0.2 to about 3.0. The coefficient of friction for nitinol may range from about 0.02 to about 0.06. This relatively low coefficient of friction may enable the drainage device 100 including ePTFE to be advanced more easily over the wire guide 160 as described above. This may make implantation of the drainage device 100 including ePTFE easier for the physician which also may reduce the time required for an implantation procedure.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

We claim:

1. A non-expandable endoluminal prosthesis for implantation within a body lumen, the prosthesis comprising:
an elongate tubular conduit having a first end segment and a second end segment, a drainage lumen extending longitudinally within the tubular conduit, and an actuating lumen extending longitudinally within the tubular conduit;
an actuating member received within the actuating lumen;
wherein the endoluminal prosthesis is movable between a delivery configuration in which the tubular conduit is substantially linear and a deployed configuration in which at least one of the first end segment and the second end segment includes a retaining mechanism configured to retain the prosthesis in place relative to the body lumen, and the actuating member is configured to urge the endoluminal prosthesis toward the deployed configuration.

2. The endoluminal prosthesis of claim 1, wherein the actuating lumen is positioned adjacent to the drainage lumen within the tubular conduit.

3. The endoluminal prosthesis of claim 2, wherein the drainage lumen is offset from a longitudinal axis of the tubular conduit.

4. The endoluminal prosthesis of claim 3, wherein the tubular conduit comprises a wall surrounding the drainage lumen, and the wall of the tubular conduit comprises a thin walled section and a thick walled section.

5. The endoluminal prosthesis of claim 4, wherein the actuating lumen is positioned within the thick walled section of the tubular conduit.

6. The endoluminal prosthesis of claim 1, wherein the retaining mechanism comprises a first retaining mechanism comprising a first loop and a second retaining mechanism comprising a second loop, the first end segment includes the first loop, and the second end segment includes the second loop.

7. The endoluminal prosthesis of claim 1, wherein the actuating member comprises a superelastic material.

8. The endoluminal prosthesis of claim 7, wherein the actuating member comprises a wire including the superelastic material and the superelastic material is nitinol.

9. The endoluminal prosthesis of claim 1, wherein the tubular conduit comprises expanded polytetrafluoroethylene.

10. A system comprising:
a non-expandable endoluminal prosthesis for implantation within a body lumen, the prosthesis comprising:
an elongate tubular conduit having an end segment, a drainage lumen extending longitudinally within the tubular conduit, and an actuating lumen extending longitudinally within the tubular conduit; and
an actuating member received within the actuating lumen, the actuating member being movable between a relaxed condition in which a longitudinal axis of the actuating member is substantially non-linear and a strained condition in which the longitudinal axis of the actuating member is substantially linear; and
a guide wire receivable within the drainage lumen of the prosthesis;
wherein, with the guide wire received within a portion of the drainage lumen corresponding to the end segment of the tubular conduit, the actuating member is in the strained condition, and, upon removal of the guide wire from the portion of the drainage lumen corresponding to the end segment, the actuating member moves to the relaxed condition to form a retaining mechanism in the end segment of the tubular conduit.

11. The system of claim 10, wherein the end segment of the tubular conduit comprises a first end segment and a second end segment positioned opposite the first end segment, the actuating member comprises a first end segment corresponding to the first end segment of the tubular conduit and a second end segment corresponding to the second end segment of the tubular conduit, and, in the relaxed condition, each of the first end segment and the second end segment of the actuating member is non-linear.

12. The system of claim 10, wherein the end segment of the tubular conduit comprises a first end segment and a second end segment positioned opposite the first end segment, the actuating member comprises a first actuating member and a second actuating member, the actuating lumen comprises a first portion corresponding to the first end segment of the tubular conduit and a second portion corresponding to the second end segment of the tubular conduit, the first actuating member is received within the first portion of the actuating lumen, and the second actuating member is received within the second portion of the actuating lumen.

13. The system of claim 10, wherein the actuating member is configured to move the endoluminal prosthesis from a delivery configuration in which the tubular conduit is substantially linear to a deployed configuration in which the end segment of the tubular conduit comprises the retaining mechanism.

14. The system of claim 13, wherein the end segment of the tubular conduit comprises a first end segment and a second end segment positioned opposite the first end segment, and, in the deployed configuration, each of the first end segment and the second end segment of the tubular conduit comprises at least one loop.

15. The system of claim 10, wherein the tubular conduit comprises expanded polytetrafluorethylene.

16. The system of claim 10, wherein the actuating member comprises a superelastic material.

17. The system of claim 17, wherein the superelastic material is nitinol.

18. The system of claim 18, further comprising an adhesive affixing the actuating member to the tubular conduit.

19. A method of implanting a non-expandable endoluminal prosthesis within a body lumen, the method comprising:
introducing the endoluminal prosthesis in a delivery configuration into the body lumen over a wire guide, the endoluminal prosthesis comprising an elongate tubular conduit having a first end segment, a second end segment, a drainage lumen extending longitudinally within the tubular conduit, and an actuating lumen extending longitudinally within the tubular conduit, an actuating member received within the actuating lumen;
removing the wire guide from the first end segment of the tubular conduit to enable at least a first portion of the actuating member corresponding to the first end segment to move to a relaxed configuration to form a first retaining mechanism in the first end segment of the tubular conduit;
removing the wire guide from the second end segment of the tubular conduit to enable at least a second portion of the actuating member corresponding to the second end segment to move to the relaxed configuration to form a second retaining mechanism in the second end segment of the tubular conduit.

20. The method of claim 19, wherein the tubular conduit comprises expanded polytetrafluorethylene.
